(19) United States
(12) Patent Application Publication
(10) Pub. No.: US 2010/0056886 A1
(43) Pub. Date: Mar. 4, 2010

(54) VITAL SIGN MONITOR SYSTEM AND METHOD

(76) Inventors: Jean Denis Hurtubise, Beloeil (CA); Daniel Tremblay, Sainte-Julie (CA); Sylvain Trottier, St-Lambert (CA)

Correspondence Address: GOUDREAU GAGE DUBUC, 2000 MCGILL COLLEGE, SUITE 2200, MONTREAL, QC, H3A 3H3, CA

(21) Appl. No.: 11/571,871

(22) PCT Filed: Jul. 8, 2005
(86) PCT No.: PCT/CA05/01064

§ 371 Date: Jan. 7, 2009

Related U.S. Application Data
(60) Provisional application No. 60/586,228, filed on Jul. 9, 2004.

(51) Int. Cl. A61B 5/1455; A61B 5/02

(52) U.S. Cl. 600/324; 600/500

(57) ABSTRACT

A portable vital sign monitor is provided which has a palm vital sign monitor unit carried by the patient, the unit comprising an optical probe positioned in the palm of the patient which measures at least one vital sign including SpO₂ and pulse rate but not exclusively and only those vital signs. The detected vital signs are stored in memory and transmitted by wireless inter-connection to a communication base unit, which transmits the vital sign by a phone line, LAN, Internet, serial interface or the like to a data processing device/centre.

The present invention claims the benefit of a commonly assigned provisional application entitled “Portable vital sign monitor”, which was filed on Jul. 9, 2004 and assigned Ser. No. 60/586,228. The entire contents of the foregoing provisional patent application are hereby incorporated by reference.

### FIELD OF THE INVENTION

The present invention relates to a vital sign monitoring system. In particular, the present invention relates to a portable vital sign monitor supported provided with an infra-red optical sensor positioned in the palm of a patient's hand for sensing vital signs such a SpO₂, pulse rate and potentially other vital signs. The detected vital signs are stored in memory for later transfer to a centralised server, for example by means of a communication base station and a interposed communication system such as a telephone line, computer network, etc.

### BACKGROUND TO THE INVENTION

Patient vital sign monitoring may include measurements of blood oxygen, blood pressure, respiratory gas, and EKG among other parameters. Each of these physiological parameters typically require a sensor in contact with a patient and a cable connecting the sensor to a monitoring device. For example a conventional pulse oximetry system used for the measurement of blood oxygen comprises a sensor, a patient cable and a monitor. The sensor is typically attached to a finger, earlobe or toe. The sensor has a plug that can be connected to a cable which in turn is connected to a socket located in the monitor. The cable transmits an LED drive signal from the monitor to the sensor and a resulting detected from the sensor to the monitor. The monitor processes the detector signal to provide, typically, a numerical readout of the patient's oxygen saturation and a numerical readout of pulse rate.

One draw back with such prior art sensors is that they are large and typically not portable. In the case that they are portable such prior art monitors are cumbersome and too heavy to be attached to a patient's wrist without severely hampering the patient's movement. Another drawback is that the positioning of the sensor on the end of a patient's ginger leads to many artefacts and other noise being introduced into the signals collected by the sensor as a result of articulation of the patient's fingers. Additionally, the positioning of the sensor on the finger tip effectively prohibits use of that finger, thereby reducing patient mobility. Still another drawback is that such portable devices do not provide wireless interconnection with other devices, such as data processing, networking and storage equipment, thereby reducing the potential of remote monitoring of a patient's condition and the like.

### SUMMARY OF THE INVENTION

The present invention overcomes the above and other drawbacks by providing a portable vital sign monitor, comfortable to the patient and mountable on either hand while at the same time minimising the amount by which use of the patient's hands are restricted by the monitor. The conventional finger tip SpO₂ monitoring (requiring a support/sensing assembly on a finger and an interconnecting cable between sensor and processing device) which greatly inhibits the use of the patient's hand has been done away with. It is also an object of the invention to provide a palm vital sign sensor in a location which allows for improved pulse detection.

Accordingly, the present invention provides a system for monitoring at least one vital sign of a patient. The system comprises a base unit comprising a wireless interface and at least one interconnection to a data processing system and a portable monitor worn or otherwise carried by a patient. The monitor comprises at least one detector for measuring at least one vital sign of the patient at predetermined intervals, a clock for providing a time of measuring the at least one vital sign, a processor for pre-processing the at least one measured vital sign according to a predefined program and predetermined configuration settings and stamping the pre-processed vital sign with the time of measuring, a memory and a wireless interface for communicating with the base unit wireless interface. When the portable monitor is within a wireless communication range of the base unit the pre-processed vital sign is relayed together with the time stamp to the base unit and wherein when the portable monitor is outside of the base unit range each of the pre-processed vital sign and the time stamp are stored in the memory, each of the stored pre-processed vital sign and the time stamp being relayed to the base unit when the monitor re-enters the range of the base unit. When the base unit relays each of the pre-processed vital sign and the time stamp to the data processing system.

There is also provided a monitor for monitoring the SpO₂ of a patient. The monitor comprises a detector comprising a first LED emitting light having a wavelength in the visible range, a second LED emitting light having a wavelength in the infrared range and a photodetector. When the first and second LEDs and the photodetector are positioned facing towards and proximate to a first metacarpal of a hand of the patient.

Additionally, there is provided a detector for use with a monitor for monitoring the SpO₂ of a patient. The detector comprises a band adapted to fit snugly around the base of a digit of the patient and having an inner surface and electronics comprising a first LED emitting light having a wavelength in the visible range, a second LED emitting light having a wavelength in the infrared range, a photodetector and a connector for interconnecting the electronics with the monitor. When the first and second LEDs and the photodetector are exposed along the inner surface and positioned such that light emitted by the LEDs is received by the photodetector.

Other objects and advantages of the invention herein will become apparent from the specification herein.

### BRIEF DESCRIPTION OF THE DRAWINGS

FIG. 1 is a schematic diagram of a wireless health monitoring system according to an illustrative embodiment of the present invention;

FIG. 2A is a top view of a portable vital sign monitor according to an illustrative embodiment of the present invention mounted on a patient's wrist;

FIG. 2B is a top view of a portable vital sign monitor according to an illustrative embodiment of the present invention;

FIG. 2C is a side plan view of a ring SpO₂ sensor in accordance with an illustrative embodiment of the present invention;

FIG. 2D is a top view of a portable vital sign monitor according to an alternative illustrative embodiment of the present invention;

FIG. 3 is a schematic diagram of the electronics of a portable vital sign monitor according to an illustrative embodiment of the present invention;

FIG. 4 is a raised front perspective view of a base unit according to an illustrative embodiment of the present invention;

FIG. 5 is a schematic diagram of the electronics of a base unit according to an illustrative embodiment of the present invention mounted on a patient's wrist;

FIG. 6 is a raised front perspective view of a RF interface module according to an illustrative embodiment of the present invention;

FIG. 7 is a schematic diagram of the electronics of a RF interface module according to an illustrative embodiment of the present invention; and

FIG. 8 is a flow chart of an illustrative embodiment of a SpO₂ data acquisition algorithm.

### DETAILED DESCRIPTION OF THE ILLUSTRATIVE EMBODIMENTS

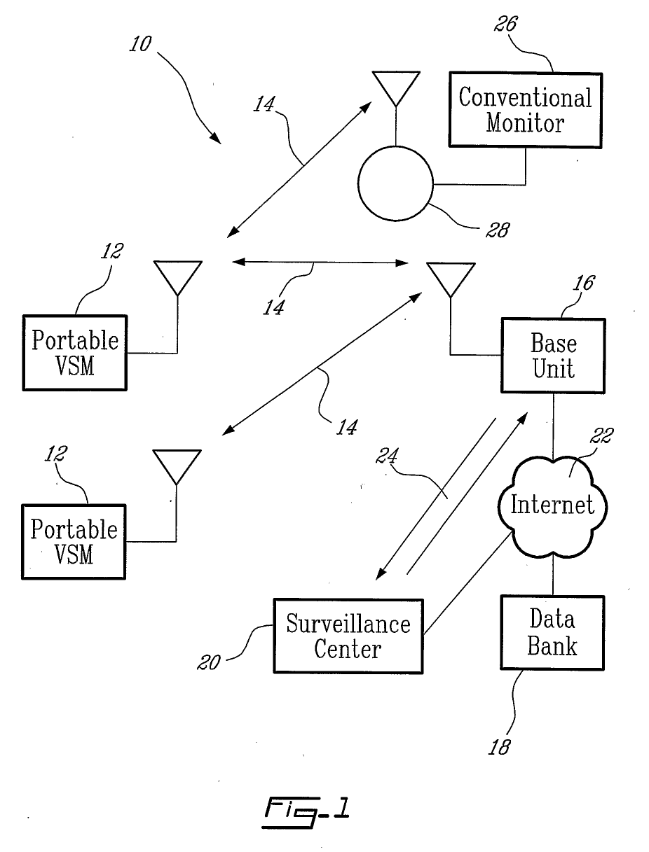

Referring now to FIG. 1, there is disclosed in accordance with an illustrative embodiment of the present invention a vital sign monitoring system, generally referred to using the reference numeral 10. The system is comprised of one or more portable vital sign monitors as in 12 which communicate via radio frequency (RF) connections as in 14 with one or more base units as in 16. The base unit 16 provides interconnection with other data processing devices, such as data banks as in 18 or other computing devices for example at a surveillance centre 20, which further process data received from the monitors as in 12 via the base unit(s) as in 16. Illustratively, the interconnection is provided via the internet 22 or alternatively via a dial up connection 24.

In an alternative embodiment the monitors as in 12 can communicate directly with a conventional vital sign monitor 26 via an RF interface module 28 or other wireless interface such as infrared or the like. In this regard, the combination of the monitor 12 and the RF interface module 28 effectively supplants the wired interconnection between the conventional vital sign monitor 26 and the patient which would otherwise be necessary.

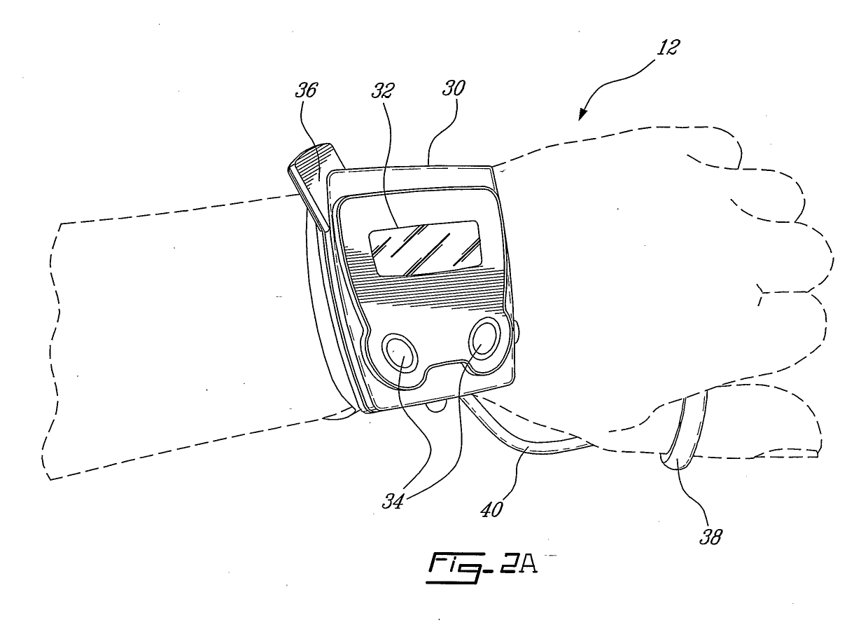

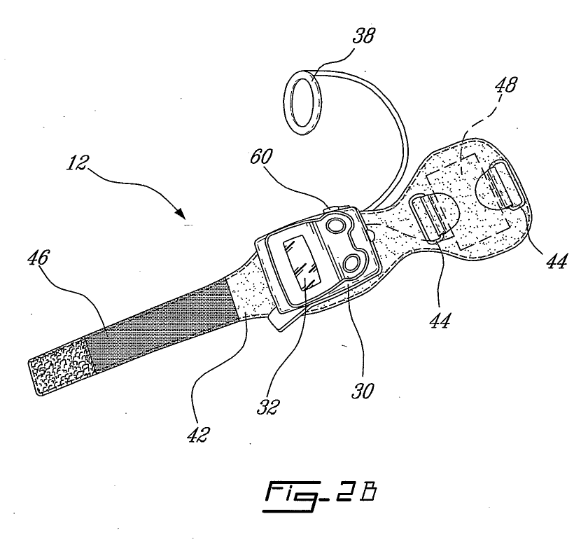

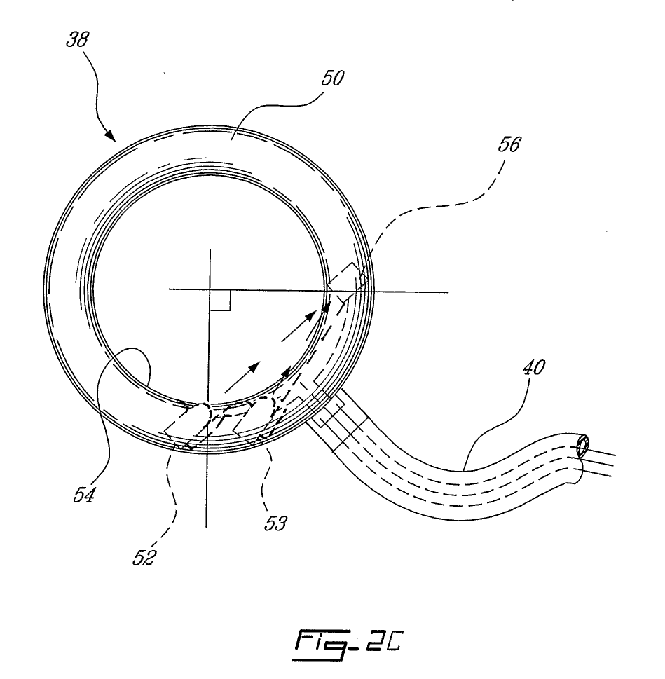

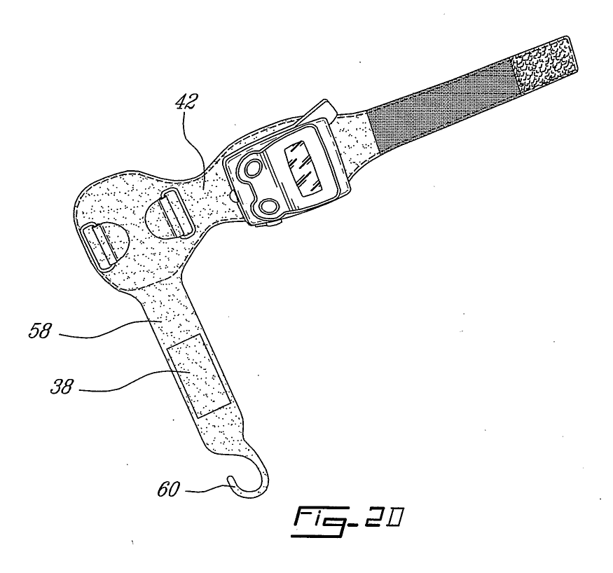

Referring now to FIG. 2A, the portable vital sign monitor 12 is mounted on the wrist of a patient and comprises a housing 30, manufactured, for example, from a rigid plastic or the like. The housing 30 encases and protects electronics (not shown) mounted therein. A display 32 is mounted on a visible outer surface of the housing 30 for displaying relevant information to the patient. A series of buttons as in 34 are also mounted on the front surface of the housing 14 allowing the internal electronics of the monitor 12 to be accessed. An antenna 36 is provided for interconnecting the monitor 12 with other wireless devices (not shown). Additionally, the monitor 12 comprises at least one detector unit 38 interconnected to the electronics housed within the housing 30 via a conductive interconnecting cable 40.

Referring now to FIG. 2B, the monitor housing 30 is secured to the patient's wrist by a wrist band 42 which is attached to a pair of suitable slots or conventional spring loaded watch wrist band mounting pins (not shown) fashioned in the under surface of the monitor housing 30. The wrist band 42 is fabricated from leather, elastic and/or woven materials such as nylon, Lycra®, Coolmax® and the like. Integral to the wrist band 42 is a series of buckles 44 and a Velcro® fastener 46 for securing the wrist band 42 to the patient's wrist. A portion of the wrist band 42 is dedicated to housing a small flat rechargeable battery 48 for supplying power to the electronics contained within the housing 30 via a pair of electrical leads (not shown). Additionally, one or more detectors (not shown), for example for extracting the pulse or the temperature of the patient, may be incorporated into the wrist band 42.

Referring now to FIG. 2C in addition to FIG. 2A, the detector unit 38 is illustratively a SpO₂ detector unit comprised of a ring, or band, 50 adapted to fit snugly around the patient's digit such as a thumb, finger or toe. Preferably the ring 50 is adapted to fit around the phalanx of the thumb, a region which combines good blood flow along with proximity to the wrist, thereby allowing a shorter interconnecting cable 40 to be used. Note that although in the illustrative embodiment an interconnecting cable 40 comprised of a series of conductive wires is shown, in a particular embodiment, and with provision of an independent source of power to the ring 50 as well as the requisite interfacing electronics, the interconnection between the monitor 12 and the electronics housed within the monitor housing 30 could also be carried out via a wireless interface (not shown), such as an infrared or RF interface.

Still referring to FIG. 2C, in order to provide the basic signals for the SpO₂ detection the detector unit 38 is illustratively a reflective sensor which combines a pair of light emitting sources as in 52, 53, one emitting light having a wavelength the visible spectrum and the other emitting light having a wavelength in the infrared spectrum, embedded into the ring 50 such that they are exposed on an inner surface 54 of the ring 50 with an optical detector 56, for example a high sensitivity phototransistor, also embedded in the ring 50 such that light incident on the inner surface 54 of the ring 50 in the region of the photodetector 56 is detected. The visible and infra-red light sources 52, 53 (such as a visible light emitting LED and an infra-red light emitting LED) generate two wavelengths of light which, after passing through the blood vessel(s) located in the patient's thumb, finger or toe, are detected by the optical detector 56. As known in the art, SpO₂ can be measured non-invasively by illuminating a region with good blood flow with a light source emitting two wavelengths of light, the first between 600 nm and 700 nm (illustratively 650 nm) and the second between 800 and 940 nm (illustratively 805 nm). As the light is partially absorbed by the haemoglobin by amounts which differ depending on whether the haemoglobin is saturated with oxygen (or not), by calculating the absorption at the two wavelengths the amount of haemoglobin which is saturated with oxygen can be calculated.

Still referring to FIG. 2C, in order to ensure adequate reception of the light by the photodetector 56, the light sources 52, 53 are illustratively positioned within a quarter turn of the photodetector 56. Additionally, the light sources 52, 53 and photodetector 56 are also illustratively positioned such that they face generally towards one another.

Referring now to FIG. 2D, in an alternative illustrative embodiment, the wrist band 42 includes a detector unit supporting portion 58 and a band 60 through which the thumb is inserted. The detector unit 38 is held flush against the heal of the thumb of a patient in the region of the first metacarpal by the detector unit supporting portion 58. Optionally, a pressure applying insert (not shown), manufactured from a flexible material such as steel, plastic or the like is provided for applying a light pressure to the back of the detector unit 38, thereby forcing it lightly against the patient's skin and improving the performance of the detector unit 38. The wrist band is configured so that, when properly secured on the thumb, the detector unit 38 overlies the major blood vessels of the palm located in the region of the first metacarpal. In this position, the sensing of movement of the blood is particularly good, as the vessels supplying blood to the palm converge and pass through this region fairly close to the inside surface of the hand, which in turn makes optical observation more effective. Additionally, articulation of the hand generally gives rise to less movement in the thumb than in the fingers and the finger tips, which will generally give rise to an improved signal quality.

Referring back to FIG. 2B, as discussed above, in order to ensure portability the monitor 12 is powered by a dedicated battery pack 48 comprised of one or more rechargeable lithium ion batteries or the like, which is inserted into a pocket integral to the wrist band 42. The battery pack 48 is interconnected with the electronics within the monitor housing 30 by a pair of electrical leads (not shown). Additionally, there is provided an external re-charger port 60 (as well as the associated electronics) allowing the monitor 12 to be interconnected with a re-charger (not shown) for recharging the battery pack 48.

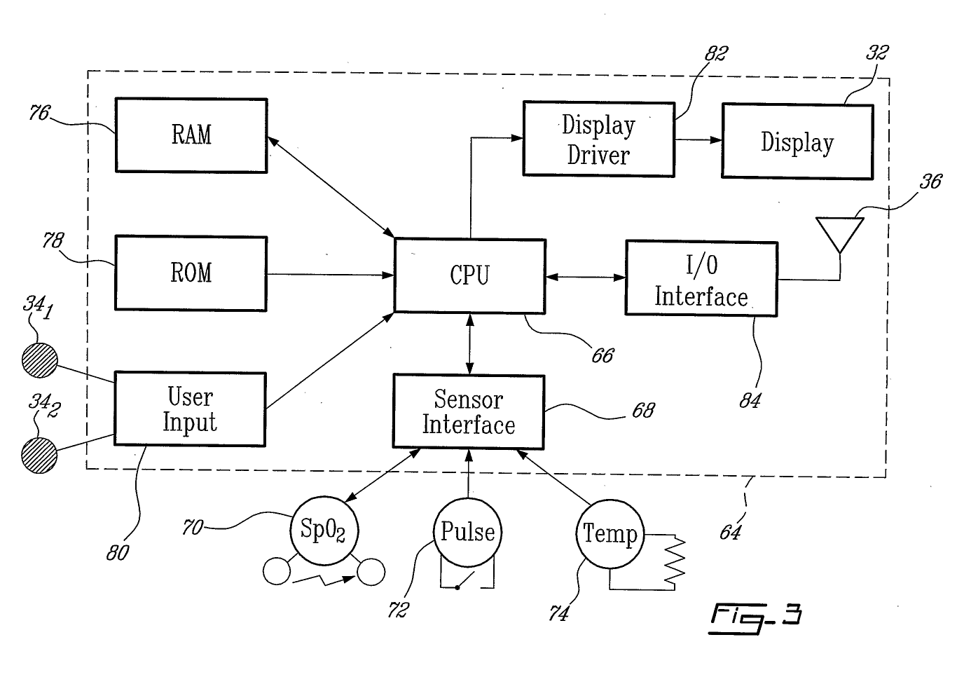

Referring now to FIG. 3, an illustrative embodiment of the electronics 64 which control the various functions of the portable vital sign monitor 12 will now be described. The heart of the electronics is a microprocessor (CPU) 66. The microprocessor 66 receives data from the sensor interface 68 which collects data from at least one sensor. Illustratively, these sensors may include a reflective sensor (SpO₂) 70 for providing the raw data for measuring SpO₂, a sensor for measuring heart rate (Pulse) 72, a sensor for measuring patient temperature (Temp) 74, etc. Illustratively, the sensor interface 68 converts readings from the sensors into digital formats readable by the microprocessor 66. The microprocessor 66 pre-processes the digitised sensor readings according to one or more programs and configuration settings stored in the ROM 76 and RAM 78 and includes the ability to store resultant values in the RAM 78. Note that although reference is made to a CPU, ROM and RAM, the use of other types of microprocessors/microcontrollers and memory devices, including but not limited to Electrically Erasable Programmable ROMs (EEPROMs), Programmable Logic Arrays (PLAs), Field Programmable Gate Arrays (FPGAs), etc., is within the scope of the present invention. Pre-processing raw data received from the sensor interface 68 according to stored programs and configuration settings reduces the amount of data which is subsequently stored in the RAM 78, for example by reducing the rate at which values are generated or eliminating erroneous readings. As will be seen below, this also reduces the amount of data which is eventually transferred from the monitor 12.

The user input interface 80 transfers the status of the one or more user input buttons as in 34 to the microprocessor 66 allowing the user to control the operation of the electronics. The microprocessor 66 is also connected to the display 32 via a display driver 82. The display 32 provides the user with useful information, such as the date and time, status, current readings, etc.

An Input/Output (I/O) interface 84 is provided allowing the electronics to communicate with external devices. Illustratively, and as discussed above, the I/O interface 84 is a RF interface which interconnects with other devices, for example the base unit 16 of FIG. 1, via an antenna 36. The RF communications signal transmitted via the antenna 36 illustratively has a frequency in the range of 400 MHz family and up pursuant to FCC regulation, part 15 and is analogous to the radio transmission used in a cordless phone. However, a variety of wireless transmission methods may be used, such as, e.g., electromagnetic transmission under 928 MegaHertz, Bluetooth®, etc. Typically, the transmission is low power and can be limited to travel of less than 100 meters. Note also that, although a RF interface is shown, other types of wireless interfaces, including for example those based on infra-red technology, could also be used in a particular embodiment. Additionally, although the I/O interface 84 has been described primarily for the transmission of vital sign data between the monitor 12 and the base unit 16, as will be seen below the I/O interface 84 can also be used, for example, by the monitor 12 to receive software updates, configuration data and other remote user inputs.

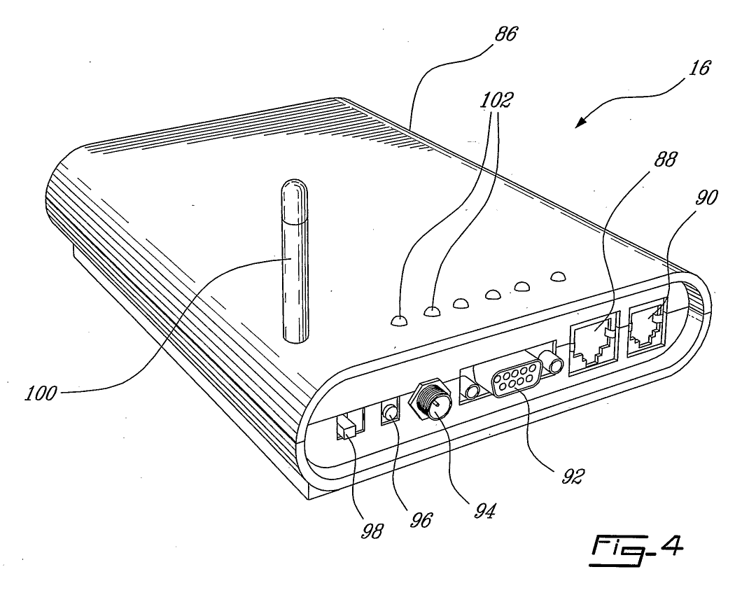

Referring now to FIG. 4 in addition to FIG. 1, each base unit 16 is comprised of a housing 86 manufactured from a durable non-conductive material such as plastic, and enclosing electronics and a rechargeable battery (both not shown). A variety of external interfaces, such as a RJ-45 jack 88, RJ-11 jack 90 and serial connector 92 (such as RS-232 or USB) are provided. Additionally, an external power supply jack 94 is provided for thereby allowing the base unit 16 to be powered by an external power supply (not shown) as well as for recharging the rechargeable battery. Additionally, a reset button 96 is provided in order to allow the user to restore factory settings, as well as an on/off switch 98. In order to communicate with the portable vital sign monitor(s) (reference 12 in FIG. 1), via RF an antenna 100 is provided. A series of status LEDs as in 102 are visible in the housing 86 and provide the user with a visual indication as to the actual status of the base unit 16.

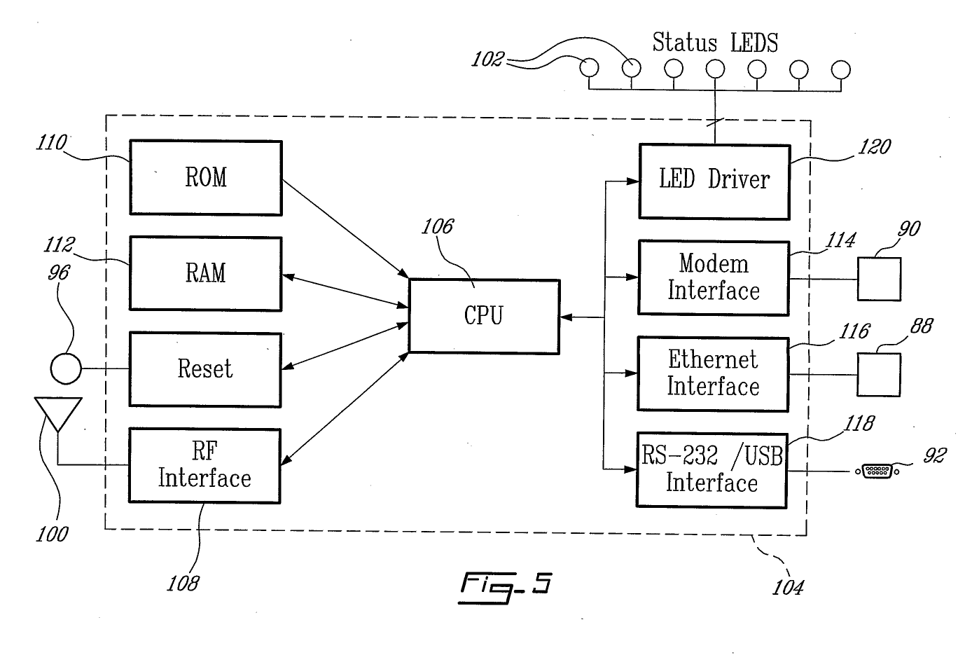

Referring now to FIG. 5 in addition to FIG. 4, in an illustrative embodiment the electronics 104 of the base unit 16 comprises a microprocessor (CPU) 106. The microprocessor 106 receives data from one or more portable monitors (reference 12 in FIG. 1) via an RF interface 108 in a prescribed format and, using one or more software programs and predefined settings stored in ROM 110 and/or RAM 112, processes the received data. Note that although reference is made to a CPU, ROM and RAM, the use of other types of microprocessors/microcontrollers and memory devices, including but not limited to Electrically Erasable Programmable ROMs (EEPROMs), Programmable Logic Arrays (PLAs), Field Programmable Gate Arrays (FPGAs), etc., is within the scope of the present invention.

Still referring to FIGS. 4 and 5, depending on predefined settings held in the ROM 110 and/or RAM 112, the microprocessor 106 is able to relay data received via the RF interface 108 to other external devices (not shown), for example via a modem interface 114 and the RJ-11 jack 90, via an Ethernet interface 116 and RJ-45 jack 88, or a RS-232/USB interface 118 and serial connector 92. The base unit 16 uses these links to primarily to relay the vital sign data received from the portable vital sign monitor 12 to, for example, a surveillance centre (not shown) or the like, although these interfaces can also be used to relay other information such as configuration settings and status of the monitor 12. Of course, a person of skill in the art will understand that each one of the modem interface 114, Ethernet interface 116 and RS232/USB interface 118 includes the necessary electronics and hardware for interconnecting with their respective communication devices/networks according to their respective standards. The microprocessor 106 is additionally able to provide, via a LED driver 120, a summary of current status via the series of status LEDs as in 102.

Still referring to FIGS. 4 and 5, the base unit 16 can be attached to an external PC or the like, illustratively via the RS 232/USB interface and serial connector 92, in order to update the one or more software programs stored in ROM 110 as well as to modify settings stored in ROM 110 and/or RAM 112. This also allows other configuration parameters, such as Ethernet address and telephone numbers to be dialled by the modem interface, to be modified. Additionally, as discussed above the settings stored in ROM 110 and/or RAM 112 can be returned to the factory default settings by activating the reset button 96.

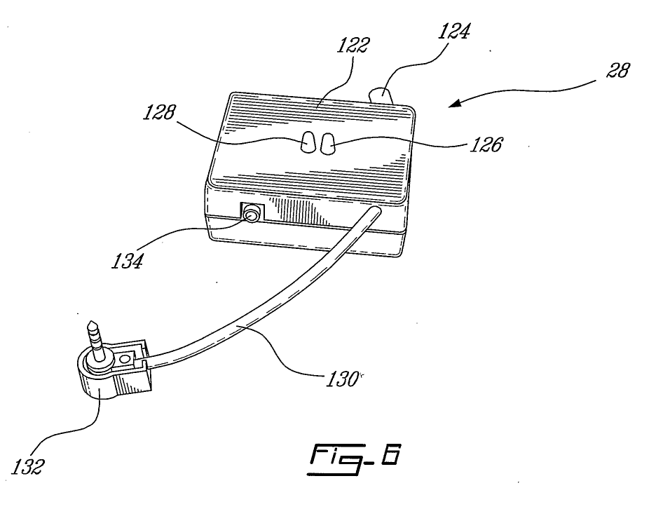

Referring now to FIG. 6, the RF interface module 28 comprises a housing 122 manufactured from a durable material such as plastic encasing electronics and a rechargeable battery (both not shown). The RF interface module 28 furthermore comprises and antenna 124 for communicating with a portable vital sign monitor (reference 12 in FIG. 1). A pair of status LEDs 126, 128 provide the user with an indication of the status of the communications between the portable vital sign monitor and the RF interface module 28. In order to attach the RF interface module 28 to a conventional vital sign monitor (reference 26 in FIG. 1), a connecting cable 130 is provided comprising a connector plug 132 adapted to match the interface (not shown) provided on the conventional vital sign monitor. Additionally, an external power supply jack 134 is provided for thereby allowing the RF interface module 28 to be powered by an external power supply (not shown) as well as for recharging the rechargeable battery.

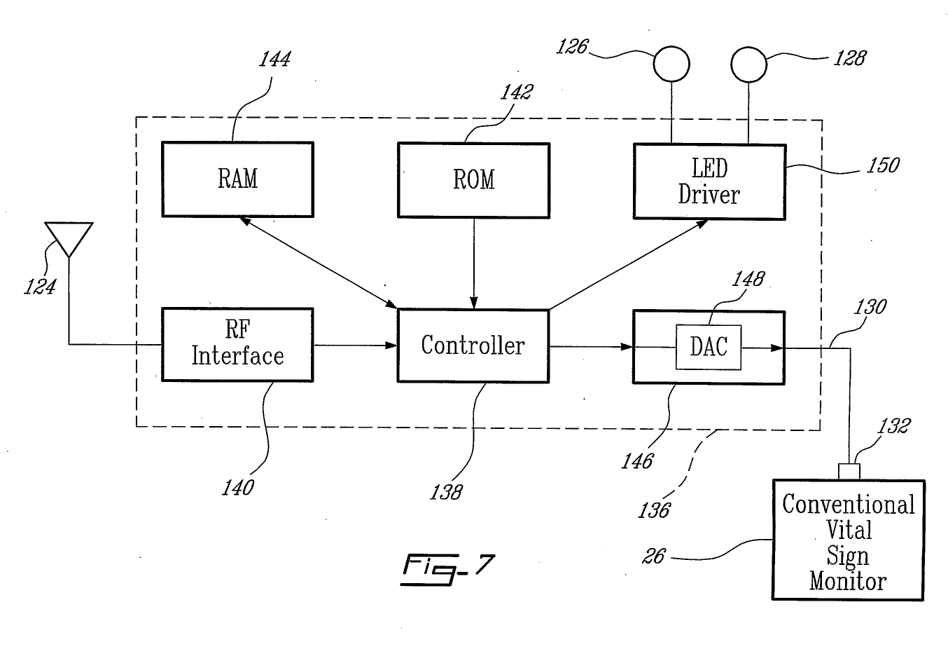

Referring now to FIG. 7 in addition to FIG. 6, the electronics 136 of the RF interface module 28 comprise a controller 138 which:
(1) receives a stream of raw digitised data from the portable vital sign monitor (reference 12 in FIG. 1) via the antenna 124 and RF interface module 140;
(2) conditions the received data according to software and (optionally) predetermined settings stored in the ROM 142 and/or RAM 144; and
(3) relays the conditioned data stream to an external interface module 146.

The external interface module 146 comprises a Digital to Analog Converter (DAC) 148, together with other electronics (not shown) which make up the external interface module 146, convert the conditioned digitised data into an analog format which is understood by the conventional vital sign monitor 26 to which the RF interface module 28 is attached via the connecting cable 130 and connector plug 132. Additionally, the controller, using software and (optionally) predetermined settings stored in the ROM 142 and/or RAM 144, provides control signals to the LED Driver module 150 for illuminating the status LEDs 126, 128 thereby providing the user an indication of the current status of operation of the RF interface module 28.

Referring back to FIG. 3, the electronics 64 and programs which are stored in the RAM 76 and ROM 78 of the portable monitor 12 provide for a number of different modes of operation, including:
Power-up
Setup
Normal/Power save
Alarm
Upgrade

These are discussed in more detail hereinbelow.

Power-Up Mode

Referring to FIGS. 1, 2B and 3, illustratively, the power up mode is automatically executed when a battery 48 is installed and connected to the electronics 64 via the pair of electrical leads. This mode triggers an initialization phase that ensures that the electronics 64 are ready to operate correctly. An illustrative algorithm for this mode is as follows:
Display 32 provides a power up ongoing message;
electronics 64 run an internal test to ensure that all components are present and functional (for example, battery level is verified and memory is checked);
electronics 64 initialize all internal components (such as display 32, RAM 76, serial number, temperature); and
the power up ongoing message is removed from the display 32 and the current date and time displayed.

Setup Mode

The Setup mode is triggered if software updates or the like are available for the monitor 12. Additionally, the Setup mode allows the current date and time to be set as well as other configuration parameters of the monitor 12.

Note that the setup mode is run remotely by the base unit 16 (not shown) and can be executed whenever necessary. However, in order for the base unit 16 to initiate the setup mode and send updated configuration data and the like it must wait for the monitor 12 to communicate with the base unit 16.

Illustratively, an algorithm for the setup mode is as follows (assuming communication between the base unit 16 and monitor 12):
During normal transmission, the monitor 12 transfers data to the base unit 16 via a RF connection 14;
the base unit 16 returns an acknowledge message to the monitor 12 and includes new parameters such as current date, time, other configuration parameters, and/or updated versions of the monitor software;
the monitor 12 ensures that the data received is valid (by checking CRC) and then sends an acknowledge to the base station;
the monitor 12 updates the date and time and stores the new configuration parameters into memory;
if an updated software version is provided for the monitor 12, then monitor 12 goes into Upgrade mode (see below);
the monitor 12 provides an indication that the configuration is complete message on the display 32; and
The monitor 12 returns to its previous mode.

Normal Mode

This normal mode is the default mode of operation when a patient is wearing the monitor 12 and has normal (within a given range) vital signs. Switching from normal mode to alarm mode (see below) can be carried out automatically or manually. In normal mode, the monitor 12 carries out its tasks while at the same time ensuring that use of the battery 48 is kept to a minimum. Functions carried out in the normal mode include:
updating of date and time on the display 32;
updating of time remaining before the next acquisition cycle;
updating of time remaining before the next transmission cycle;
If patient is no longer wearing the monitor 12, calculation of the time left before triggering a “no patient” alarm;
sensing the status of the monitor alarm button; and
sensing the status of the monitor select button.

While maintaining these minimum tasks, all unused circuitry (such as the vital signs sensors 70, 72, 74, sensor interface 68, I/O interface 84, display 32 and CPU 66) are placed in a power save mode. If an event from the list above occurs, then the appropriate circuitry is activated and one or more of the following tasks carried out:
Acquisition time is reached:
   an acquisition symbol is displayed on the display 32;
   the sensors 70, 72, 74 and sensor interface 68 are activated;
   vital signs are acquired and stored in RAM 76;
   if patient motion is detected during data acquisition, then a motion symbol is displayed;
   values of the acquired vital signs are compared with their acceptable ranges;
   if acquired vital signs are outside of acceptable ranges, then the alarm mode (see below) is activated;
   acquired vital signs are stored in memory, along with any specific alarm and/or status information; and
   the acquisition symbol is removed from the display 32.
Transmission time is reached:
   a transmission symbol is displayed;
   the I/O interface 84 is activated and a connection with the base unit 16 via an RF connection 14 established;
   data (vital signs, alarm and status) available in RAM 76 is transferred to the base unit 16 via the I/O interface 84;
   base unit 16 returns an acknowledge message;
   if the acknowledge message received from the base unit 16 contains new parameters such as date and time or other configuration parameters, then the monitor 12 enters the setup mode (as discussed above); and
   the transmission symbol is removed from the display 32.
Alarm button is pressed:
   the alarm mode is activated.
Select button is pressed:
   if a message other than date and time is displayed on the display 32, the message is removed; and
   if alarm mode is active, then it is deactivated.

Alarm Mode

The alarm mode is either automatically triggered by the monitor 12 when a vital sign value is outside the determined limits or triggered manually by the patient through the use of the alarm button. Manual alarms can be terminated by pressing the select button while automatic alarms are terminated automatically. An audible warning, for example intermittent buzzer sound, is provided when a manual alarm has been activated by depressing the button. Illustratively, the algorithm for this mode is as follows:
an alarm symbol is displayed on the display;
the portable unit activates the audible warning, vital sign data is acquired and stored in memory and the acquired vital sign data is transferred to the base station;
if the alarm is manual and the select button is depressed, then the alarm symbol is removed from the display 32, the audible warning is terminated and the monitor 12 returns to its previous mode; and
if the alarm is automatic and the select button is depressed, the audible alarm is cancelled. However, vital sign data continues to be acquired and stored in memory and the acquired vital sign data is transferred to the base station up until such time as the vital sign data returns to an acceptable range.

Upgrade Mode

The upgrade mode allows the software stored in the ROM 78 and/or RAM 76 to be updated. Illustratively, an algorithm for this mode is has follows:
using the internal boot loader, stored software is replaced with newly received updated software; and
the monitor 12 reenters the power up mode.

Display

Illustratively, the monitor 12 is equipped with a display 32 comprised of a LCD screen that can, for example, display up to sixteen (16) characters (2 lines of eight (8) characters, each with a 5×7 dots resolution). Illustratively, and in order to minimize the energy consumption from the battery, power to the display 32 is managed according to the following algorithm:
if the monitor 12 is idle for more than twenty (20) seconds, the display 32 will be deactivated;
if the select button is depressed while the display 32 is deactivated, then the display 32 is activated;
the display 32 is automatically activated at the beginning of an acquisition or transmission sequence; and
the display 32 is automatically deactivated at the end of an acquisition or transmission sequence.

Internal Clock

The current date and time is maintained by the electronics 64. Current date and time is used in order to timestamp any acquired vital sign data. Additionally, the current date and time can be displayed on the display 32. Typically, the date and time are displayed on the display unless there are other messages for display.

The date and time is set remotely via the base station. Illustratively, the date and time evolve automatically based on the standard Gregorian calendar (taking into account appropriate number of days per month, leap year . . . ). Additionally, such features as time zones and daylight savings time can are managed remotely.

Audible Warnings

The portable unit produces audible warnings, for example through the use of a piezoelectric buzzer or the like. The generated warning is controlled under a beep/silence sequence and frequencies. Referring to Table 1, the examples of sequences used in association with a particular event are described:

TABLE 1

| Event | Sound sequence |
|---|---|
| Manual Alarm is | 3 beeps of ½ second, each separated by |
| activated | 1 silence of ½ second (total 3 seconds) |
| | 10 silences of ½ second (total 5 seconds) |
| | This sequence is repeated continuously |
| | during all the time of the event. |
| | The total time for one cycle is 8 seconds. |
| While in acquisition | 4 beeps of 1/16 second, each separated by |
| mode, patient motion or | 1 silence of 1/16 second (total 500 ms) |
| no patient is detected for | 48 silences of 1/16 second (total 3 seconds) |
| more than 1 minute 4 | This sequence is repeated continuously |
| seconds (see acquisition | for 16 seconds. |
| algorithm). | The total time for one cycle is 3.5 seconds. |

Data Acquisition

The monitor 12 is equipped with sensors for acquiring one or more of a patient's vital signs, such as:
pulse rate;
SpO₂;
skin temperature;
blood sugar; and/or
blood pressure.

Vital sign acquisition is made under the following conditions:
the data acquisition frequency is based on a value provided during setup mode;
there are two possible data acquisition frequencies, one for normal mode and one for alarm mode;
a timestamp is recorded with each data acquisition cycle;
the data collected is kept in memory at least until its transmission to the base station has been carried out; and
the acquired vital signs data can be either full (each reading is kept) or preprocessed (for example, only the average of vital signs values is kept along with maximum, minimum peak and associated timestamps).

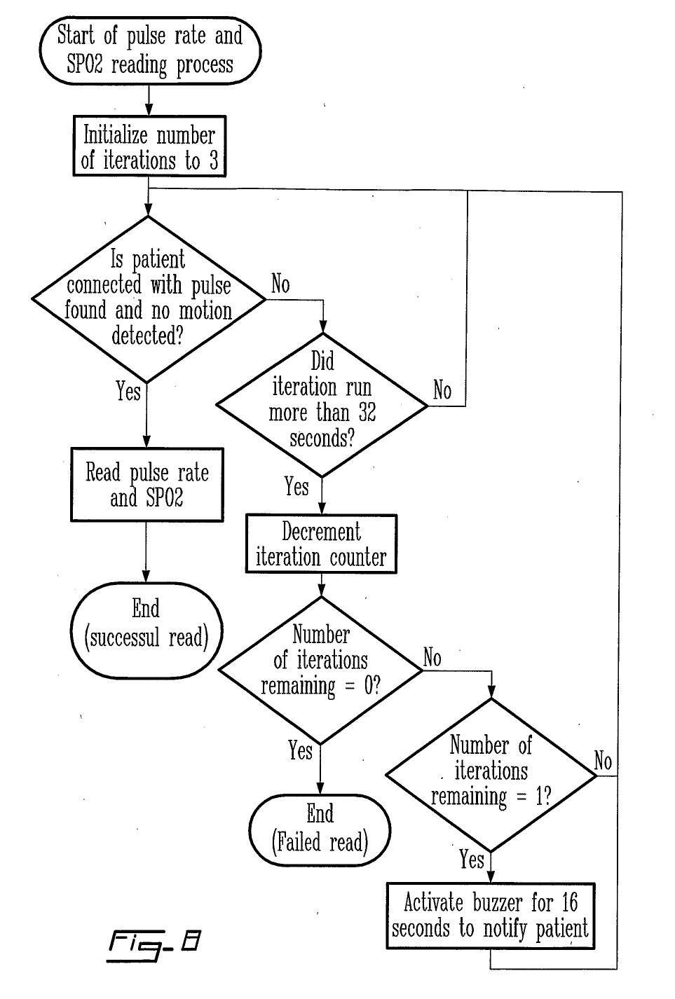

Referring to FIG. 8, an illustrative embodiment of a data acquisition algorithm for pulse rate and SpO₂ is shown.

Although the present invention has been described hereinabove by way of an illustrative embodiments thereof, these embodiments can be modified at will without departing from the spirit and nature of the subject invention.

We claim:

1. A system for monitoring at least one vital sign of a patient, the system comprising:
a base unit comprising a wireless interface and at least one interconnection to a data processing system; and
a portable monitor worn or otherwise carried by a patient, said monitor comprising:
at least one detector for measuring at least one vital sign of the patient at predetermined intervals;
a clock for providing a time of measuring said at least one vital sign;
a processor for pre-processing said at least one measured vital sign according to a predefined program and predetermined configuration settings and stamping said pre-processed vital sign with said time of measuring;
a memory; and
a wireless interface for communicating with said base unit wireless interface;
wherein when said portable monitor is within a wireless communication range of said base unit said pre-processed vital sign is relayed together with said time stamp to said base unit and wherein when said portable monitor is outside of said base unit range each of said pre-processed vital sign and said time stamp are stored in said memory, each of said stored pre-processed vital sign and said time stamp being relayed to said base unit when said monitor re-enters said range of said base unit;
wherein said base unit relays each of said pre-processed vital sign and said time stamp to said data processing system.

2. The system of claim 1, wherein said at least one detector measures a patient's vital sign selected from the group consisting of SpO₂, pulse and body temperature and combinations thereof.

3. The system of claim 1, wherein said monitor comprises a first detector for detecting a pulse of the patient and a second detector comprising a first LED emitting light having a wavelength in the visible range, a second LED emitting light having a wavelength in the infrared range, a photodetector for sensing light emitted by said first LED and said second LED.

4. The system of claim 3, wherein said first and second LEDs and said photodetector are positioned facing towards and proximate to a first metacarpal of a hand of the patient.

5. The system of claim 3, wherein said monitor further comprises a wristband for attaching said monitor to the patient and wherein said pulse detector is mounted on an inner surface of said wristband.

6. A monitor for monitoring the SpO₂ of a patient, the monitor comprising:
a detector comprising a first LED emitting light having a wavelength in the visible range, a second LED emitting light having a wavelength in the infrared range and a photodetector;
wherein said first and second LEDs and said photodetector are positioned facing towards and proximate to a first metacarpal of a hand of the patient.

7. The monitor of claim 6, wherein said first LED emits visible light having a wavelength of between about 600 nm and 700 nm and said second LED emits infra-red light having a wavelength of between about 800 nm and 940 nm.

8. The monitor of claim 7, wherein said first LED emits visible light having a wavelength of about 650 nm and said second LED emits infra-red light having a wavelength of about 805 nm.

9. The monitor of claim 6, further comprising a wristband adapted for encircling a wrist of the patient.

10. The monitor of claim 9, further comprising a band adapted to encircle a thumb of the patient and a supporting portion suspended between said wristband and said thumb band and wherein said detector is held proximate to the first metacarpal by said supporting portion.

11. The monitor of claim 9, wherein said wrist band further comprises a pocket for holding a battery.

12. A detector for use with a monitor for monitoring the SpO₂ of a patient, the detector comprising:
a band adapted to fit snugly around the base of a digit of the patient and having an inner surface; and
electronics comprising:
a first LED emitting light having a wavelength in the visible range;
a second LED emitting light having a wavelength in the infrared range;
a photodetector; and
a connector for interconnecting said electronics with the monitor;
wherein said first and second LEDs and said photodetector are exposed along said inner surface and positioned such that light emitted by said LEDs is received by said photodetector.

13. The detector of claim 12, wherein said connector comprises a series of conductive wires between the monitor and said electronics.

14. The detector of claim 12, wherein said connector comprises a wireless link between the monitor and said electronics.

15. The detector of claim 12, wherein the digit is the thumb.

16. The detector of claim 12, wherein said band is a ring formed of a rigid material.

17. The detector of claim 12, wherein a size of said band can be adjusted to accommodate different digit sizes.
